(19) United States
(12) Patent Application Publication
(10) Pub. No.: US 2026/0295269 A1
(43) Pub. Date: Oct. 1, 2026

(54) METHOD AND DEVICE FOR MEASURING MOTOR THRESHOLD AND ACTIVATION AREA FOR USE WITH PELVIC HEALTH NEUROSTIMULATION

(71) Applicants: Regents of the University of Minnesota, Minneapolis, MN (US); University of St. Thomas, St. Paul, MN (US)

(72) Inventors: Dwight E. Nelson, Minneapolis, MN (US); Gerald W. Timm, Minneapolis, MN (US); Nissrine Nakib, Minneapolis, MN (US); Sanaz Hosseini, Minneapolis, MN (US); Heather Orser, St. Paul, MN (US); Lydia G. Wilk, St. Paul, MN (US)

(21) Appl. No.: 19/576,755

(22) Filed: Mar. 24, 2026

Related U.S. Application Data
(60) Provisional application No. 63/780,853, filed on Mar. 31, 2025.

(51) Int. Cl. A61N 1/36

(52) U.S. Cl. CPC A61N 1/36139; A61N 1/36062

(57) ABSTRACT

A system includes a stimulation controller configured to provide a stimulation signal to a patient and a pressure sensor array, configured to be positioned adjacent the patient and to provide a sensed pressure value. A processor configured to associate the sensed pressure value with the stimulation signal.

### CROSS-REFERENCE TO RELATED APPLICATION

The present application is based on and claims the benefit of U.S. provisional patent application Serial No. 63/780,853, filed Mar. 31, 2025, the content of which is hereby incorporated by reference in its entirety.

### BACKGROUND

Sacral neuromodulation (SNM) also known as sacral nerve stimulation involves placing a lead having one or more electrical contacts near the sacral nerve. Electrical pulses are then sent through the lead causing the sacral nerve to fire. These electrical signals are known as stimulation signals.

A system includes a stimulation controller configured to provide a stimulation signal to a patient and a pressure sensor array, configured to be positioned adjacent the patient and to provide a sensed pressure value. A processor configured to associate the sensed pressure value with the stimulation signal.

In accordance with a further embodiment, a method includes placing a pressure sensor array adjacent a patient and applying a stimulation signal to a lead in contact with the patient. A signal from the pressure sensor array is received and at least one of the stimulation signal and a position of the lead is adjusted based on the received signal from the pressure sensor array.

In accordance with a still further embodiment, a system includes an array of pressure sensors, each pressure sensor in the array providing a respective pressure signal and a stimulation controller configured to provide a stimulation signal to a patient. A processor in the system is configured to identify a change in one of the respective pressure signals that is due to the stimulation signal.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a perspective view of a pressure sensor array mounted on a saddle-shaped support.

FIG. 2 is a front view of the pressure sensor array of FIG. 1.

FIG. 3 is a side view of the pressure sensor array of FIG. 1.

FIG. 4 is a top view of the pressure sensor array of FIG. 1.

FIG. 5 is a top view of a second embodiment of a pressure sensor array mounted on a conical support.

FIG. 6 is a side view of the pressure sensor array of FIG. 5.

FIG. 7 is a side view of a pressure sensor array in the form of a pressure mat.

FIG. 8 is a top view of the pressure sensor array of FIG. 7.

FIG. 9 is a side view of a pressure sensor array with a raised center and planar sides.

FIG. 10 is a top view of the pressure sensor array of FIG. 9.

FIG. 11 is a perspective view of a sensor array embedded in an elastic-containing garment.

FIG. 12 is a block diagram of a system used to assess and adjust the location of a stimulation lead in a patient and/or adjusting the magnitude of the stimulation signal applied to the lead.

FIG. 13 is a flow diagram of a method of obtaining pressure data before adjusting the location of a stimulation lead and/or adjusting the magnitude of the stimulation signal applied to the lead.

FIG. 14 is a graph of a stimulation signal consisting of a series of five pulses.

FIG. 15 is a flow diagram of a method of adjusting the location of a stimulation lead and/or adjusting the magnitude of the stimulation signal applied to the lead.

FIG. 16 is a flow diagram for generating a response curve.

FIG. 17 is a graph of various response curves.

FIG. 18 is a block diagram of an alternative system used to assess and adjust the location of a stimulation lead in a patient and/or adjusting the magnitude of the stimulation signal applied to the lead.

### DETAILED DESCRIPTION

Sacral nerve stimulation requires accurate placement of the stimulation lead so that the correct nerves are stimulated and so that other nerves are not stimulated. Currently, accurate placement of the lead is based substantially on verbal assessment of patient sensations and visual assessment of the lead location and/or visual assessment of muscle contractions. Such assessments are qualitative but not quantitative and as such are time consuming, incomplete, and inconsistent.

In accordance with the various embodiments, pressure sensors are provided that sense muscle contractions that are in response to stimulation signals applied to a stimulation lead. In accordance with one embodiment, the pressure sensors form a sensor array that is distributed across the exterior of a patient’s body. The sensor array can be embedded in clothing that contains elastic fiber to press the sensors against the patient’s body or can be located in a pad that the patient sits or lies upon. Each pressure sensor in the array is associated with an anatomical location on the patient and a determination of which pressure sensors in the sensor array are experiencing a pressure change can be used to determine if the stimulation lead is properly placed within the patient. Further, changes in the pressure values due to changes in the magnitude of the stimulation signal are used to construct response curves that are compared to desired response curves to determine if the lead is properly located or if therapy programming is optimal.

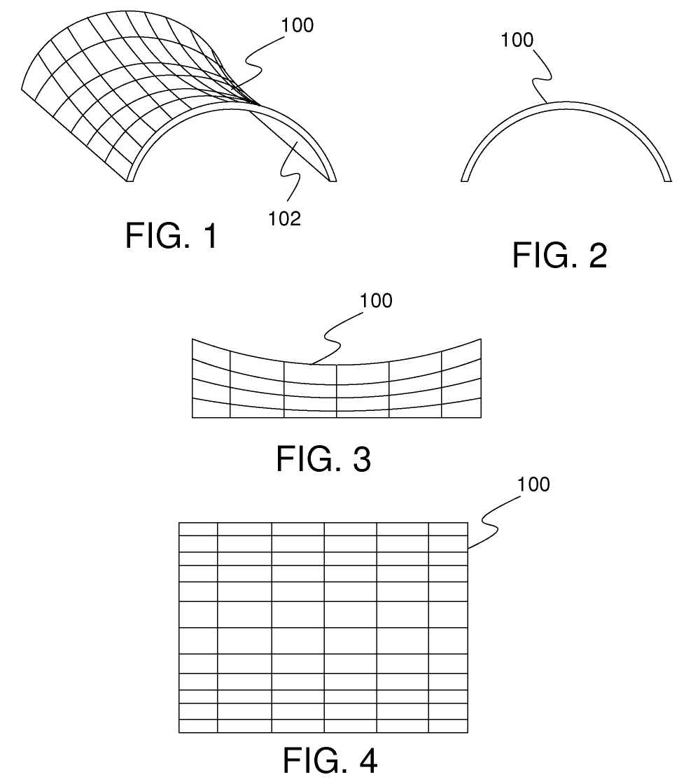

FIGS. 1-4 provide a perspective view, a front view, a side view, and a top view, respectively, of a pressure sensor array 100 in accordance with one embodiment. Pressure sensor array 100 is positioned on top of a saddle-shaped structure 102, which is able to support the weight of a patient when the patient sits on pressure sensor array 100. As shown in FIGS. 1, 3 and 4, pressure sensor array 100 has a grid appearance with each rectangle in the grid representing a single pressure sensor in pressure sensor array 100. Other spatial distributions for the pressure sensor array are possible including distributions in which the pressure sensors are not evenly distributed.

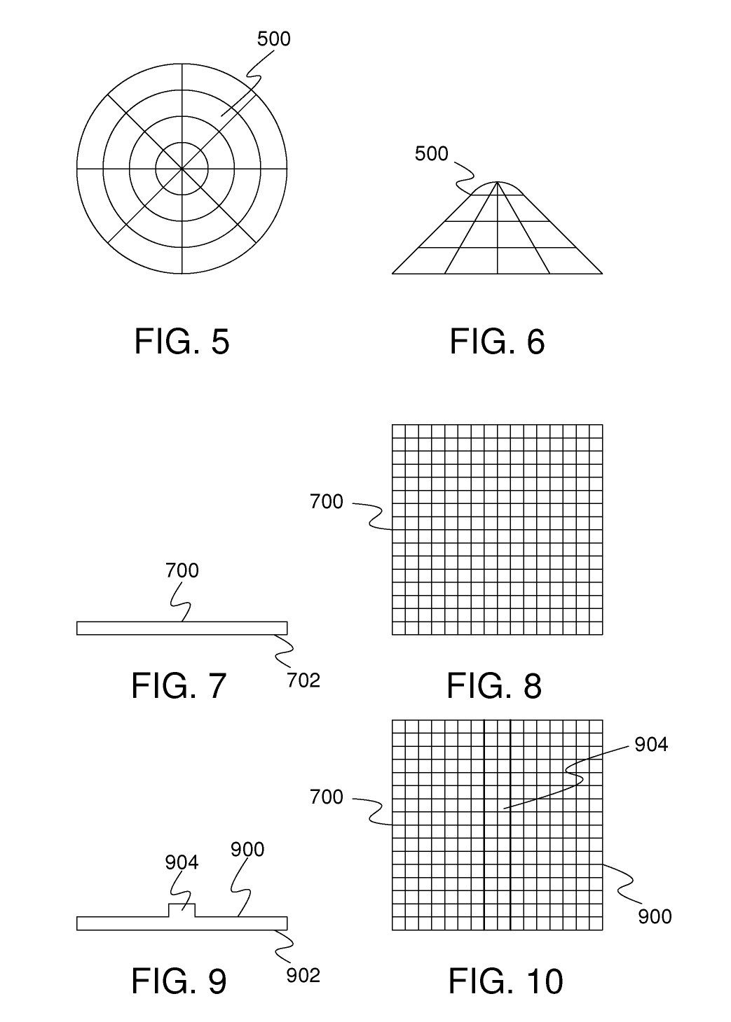

FIGS. 5 and 6 provide a top view and side view of a second embodiment showing a pressure sensor array 500, which is placed on top of a rounded conical structure that is sufficiently strong to support the weight of a patient when the patient sits on pressure sensor array 500. The lines in FIGS. 5 and 6, delimit locations of individual pressure sensors of pressure sensor array 500.

FIGS. 7 and 8 show a side view and a top view, respectively, of a third embodiment showing a pressure sensor array 700 positioned within a pad 702 that can be placed on a surface. The patient then sits or lies on pad 702. The lines shown in FIG. 8 delimit locations of individual pressure sensors of pressure sensor array 700. In some embodiments, pad 702 is rigid while in other embodiments pad 702 is flexible.

FIGS. 9 and 10 show a side view and a top view, respectively, of a pressure sensor array 900 formed on the top of a pad 902 having a raised center 904 extending the length of pad 902. During use, the patient sits on pressure sensor array 900 such that one leg is on each side of raised center 904. In FIG. 10, each area delimited by lines represents the location of an individual pressure sensor of pressure sensor array 900.

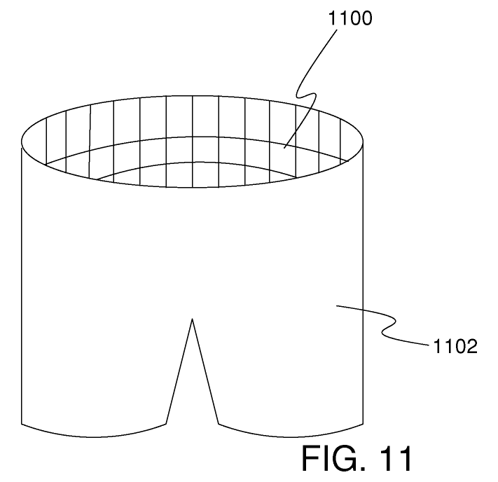

FIG. 11 provides an embodiment in which a pressure sensor array 1100 is embedded in a garment 1102 having elastic fibers that press pressure sensor array 1100 into contact with a patient when the patient wears the garment. In accordance with one embodiment, pressure sensor array 1100 is only positioned along the posterior side of the patient. In FIG. 11, the areas delimited by the lines along the interior of the garment represent locations where individual pressure sensors of pressure sensor array 1100 are positioned.

In FIGS. 1-11, electrical leads are connected to each of the pressure sensors in the respective pressure sensor arrays to allow a pressure signal to be transmitted from each pressure sensor. The pressure signals may be transmitted from the pressure sensor array wirelessly through a wireless connection within the pressure sensor array or may be transmitted through one or more wires connected to the pressure sensor array. In addition, power is provided to the pressure sensors either through a battery mounted in the same housing as the pressure sensor array or through a wired connection to a power source.

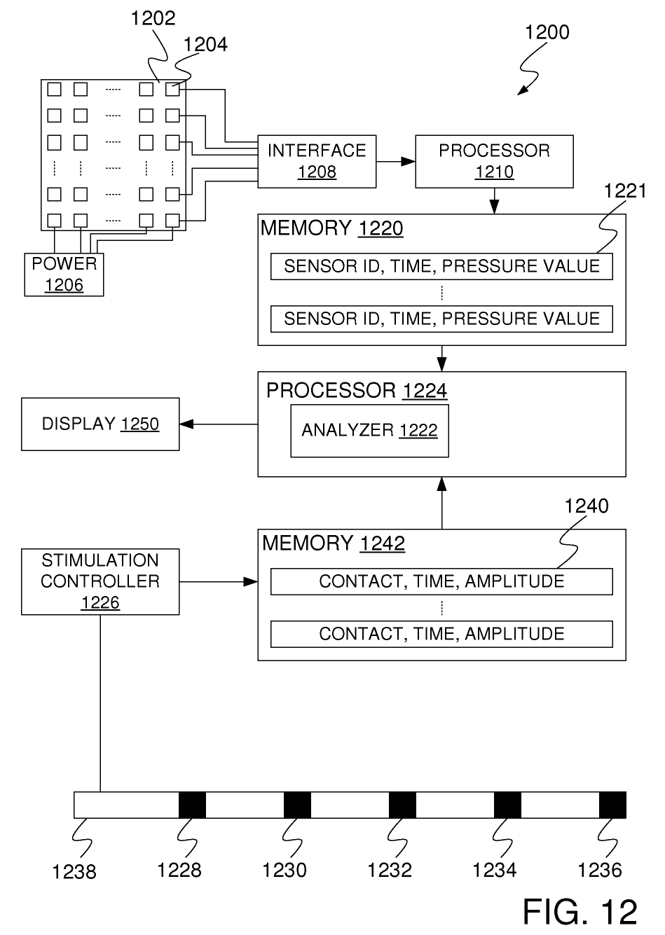

FIG. 12 provides a block diagram of a system 1200 for assessing and adjusting the location of an implanted lead in a patient. System 1200 includes a pressure sensor array 1202 consisting of a plurality of pressure sensors, such as pressure sensor 1204, where each pressure sensor provides a series of pressure values over time. Each pressure sensor receives power from a power source 1206 that can include a battery or a connection to a power line. Each pressure sensor in pressure sensor array 1202 is connected to an interface 1208 that periodically samples the pressure value provided by each pressure sensor and provides the identity of the sensor, its pressure value, and a time of the sampling to a processor 1210. The time of the pressure sampling may be synchronized to the stimulation delivery to ensure the pressure changes are due to the stimulation and not movement artifacts or that the time course for the pressure changes are correctly associated with optimal therapeutic stimulation. Interface 1208 and processor 1210 may be incorporated within the same housing as sensor array 1202 or may be separate from the sensor array housing. Processor 1210 stores the sensor ID, sampling time and pressure value for each sensed pressure value in a respective sensed pressure entry, such as sensed pressure entry 1221, in a memory 1220. To provide the sampling time, interface 1208 uses a clock (not shown).

The pressure values in memory 1220 can be accessed by an analyzer 1222 executing on a processor 1224. Processor 1224 may be the same as processor 1210 or may be different from processor 1210.

A stimulation controller 1226 is connected to the plurality of contacts 1228, 1230, 1232, 1234 and 1236 on a stimulation lead 1238 that is implanted within the patient. Stimulation controller 1226 applies stimulation signals to one or more of contacts 1228, 1230, 1232, 1234 and 1236. In addition, stimulation controller 1226 records a stimulation entry 1240 in a memory 1242 for each signal that is applied, where each stimulation entry 1240 includes an identity of the contact that received the stimulation signal, a time at which the stimulation signal was provided, and an amplitude of the stimulation signal. Memory 1242 and memory 1220 may be separate hardware devices or may be different parts of a single hardware device. Analyzer 1222 is also able to access stimulation entries 1240 of memory 1242.

Alternatively, processor 1224 can generate and store the stimulation entries in memory 1242 and then stimulation controller 1226 can use the stimulation entries as instructions for sending stimulation signals to the contacts 1228, 1230, 1232, 1234 and 1236. Thus, the contact, the time and the magnitude of the stimulation signal that is to be provided is set by processor 1224 and then is executed by stimulation controller 1126.

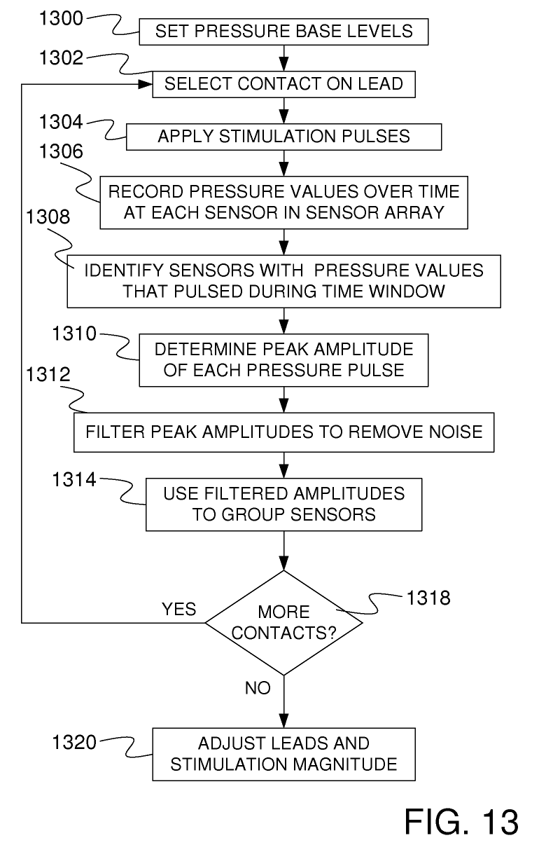

FIG. 13 provides a flow diagram of a method of assessing and adjusting the location of lead 1238 and/or adjusting the contact that the stimulation signal is sent to and/or the magnitude of the stimulation signals applied to the contacts on lead 1238.

In step 1300, base pressure levels are set for each of the pressure sensors in pressure sensor array 1202 to “zero out” each pressure sensor. These base sensor levels are set by having the patient sit or lay on the pressure sensor array or put on the garment containing the pressure sensor array while no stimulation is provided to the stimulation lead. In accordance with one embodiment, a separate base pressure value is determined for each pressure sensor by determining the average pressure value generated by the pressure sensor over a period of time while no stimulation signal is provided to the lead. This average value is then used as a “zero” value for the pressure sensor.

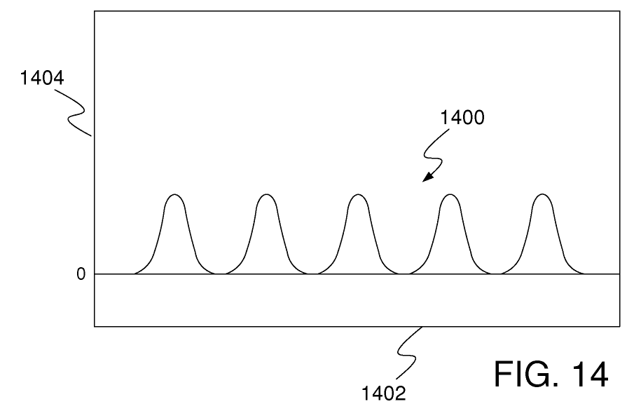

At step 1302, analyzer 1222 selects one of the lead contacts and at step 1304, analyzer 1222 instructs stimulation controller 1226 to apply a series of stimulation pulses to the selected contact. FIG. 14 provides a graph of a series of stimulation pulses with time shown along horizontal axis 1402 and amplitude shown along vertical axis 1404. In accordance with one embodiment, each pulse in the series of pulses 1400 has the same amplitude and shape. Although one particular shape is shown for the pulses in FIG. 14, in other embodiments, other pulse shapes are used.

At step 1306, pressure values from pressure sensor array 1202 are recorded in memory 1220 over a period of time with each pressure value containing a pressure sensor identifier and a time at which the pressure value was detected.

At step 1308, pressure sensors that provided a series of sensed pressure pulses are identified. In particular, in step 1308, each pressure sensor is individually selected and all of the pressure values for that pressure sensor over the period of time are retrieved from memory 1220. The pressure values are then examined to determine if the sequence of pressure values represents pressure pulses that correspond to the pulses in the stimulation signal applied to the selected contact. In accordance with one embodiment, multiple pressure sensors in pressure sensor array 1202 will provide pressure values containing pressure pulses.

At step 1310, for each pressure sensor that provided pressure pulses, the peak amplitude of each pulse in the series of pulses provided by that pressure sensor is determined. Thus, each pulse will have a separate peak value. At step 1312, the peak values determined for a pressure sensor are used to remove noise from the pressure sensor signal. In particular, the peak amplitudes are averaged for the pressure sensor to produce a filtered peak amplitude for that pressure sensor. By averaging the peak amplitudes or other time courses of the pressure changes, some of the noise in the peak amplitudes or other time courses are removed. Each pressure sensor that provided pressure pulses will have a separate filtered peak amplitude or other time courses at step 1312.

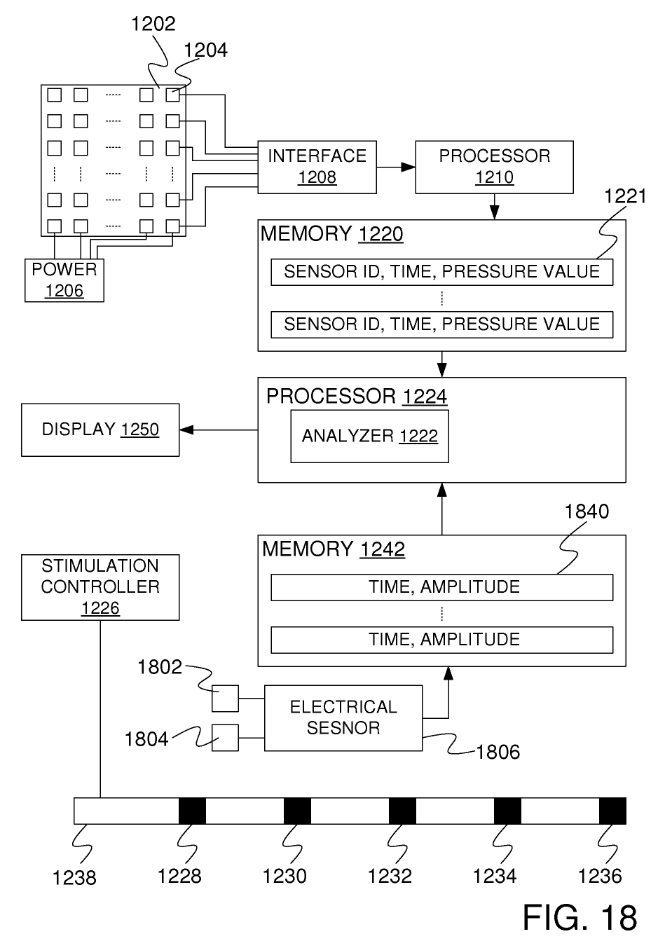

In an alternative embodiment, instead of searching for sensed pressure pulses, an additional sensor system is used to identify when the stimulation signals are applied to the patient. FIG. 18 provides a block diagram of such an embodiment where two electrical contacts 1802 and 1804 are affixed to the exterior of the patient at two spaced-apart locations. Each electrical contact conveys an electrical signal, such a voltage, present at the respective location on the patient. An electrical sensor 1806 receives the electrical signals from contacts 1802 and 1804 and generates a sequence of electrical values, such as a sequence of voltage differences between the contacts, over a period of time. The sequence of electrical values are stored as entries 1840 in memory 1242.

In this alternative embodiment, analyzer 1222 identifies times when a pulse is present in the electrical values stored in memory 1242. For each pulse in the electrical values, analyzer 1222 identifies a peak amplitude for each pressure sensor. The peak amplitudes for each pressure sensor for each pulse in the electrical values are then averaged together to provide a filtered peak amplitude for each pressure sensor. The filtered peak amplitude of each pressure sensor will have less noise than the individual peak amplitudes associated with each pulse in the electrical values.

At step 1314, analyzer 1222 uses the filtered peak amplitudes or other time courses to group pressure sensors together. There are several ways in which the pressure sensors can be grouped. In one embodiment, pressure sensors that have a filtered peak amplitude above a threshold and that are physically next to each other in the pressure sensor array are grouped together. In such an embodiment, pressure sensors that do not have filtered peak amplitudes above the threshold are not placed in a group and form boundaries between the groups. In other embodiments, group centers are identified by identifying those pressure sensors with the largest filtered peak amplitudes. For instance, in one embodiment, the three largest filtered peak amplitudes are identified and the corresponding pressure sensors are set as the respective centers of three different groups. Pressure sensors positioned around these center sensors are then added to the group. In accordance with one embodiment, isolated pressure sensors that have filtered peak amplitudes above the threshold but are not grouped with other pressure sensors can be identified and ignored for further analysis. This helps remove noisy signals in the pressure sensor array.

At step 1318, analyzer 1222 determines if there are more contacts on the lead to be tested. If there are more contacts to be tested, the process returns to step 1302 where a new contact is selected and steps 1304-1316 are repeated for the new contact. When all of the contacts have been processed, the stimulation is adjusted at step 1320 based on the values sensed by the pressure sensor array.

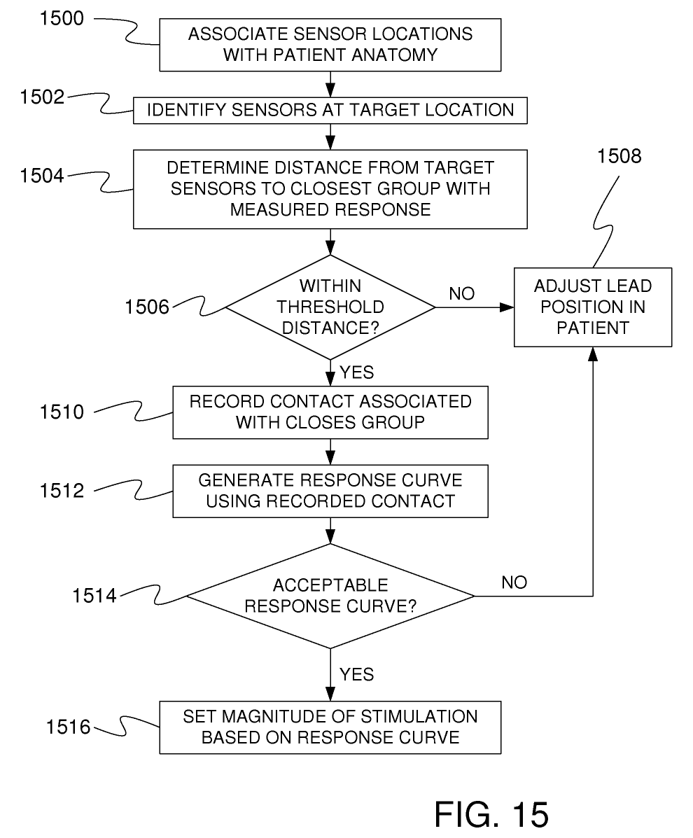

FIG. 15 provides a flow diagram of a method of adjusting the position of a lead and/or setting the magnitude or other parameters of the stimulation signal such as contact selections, frequency or pattern of pulse delivery, pulse width, or shape of the pulse in accordance with one embodiment. In step 1500, the locations of pressure sensors in the pressure sensor array are associated with the patient’s anatomy. This association is performed in one embodiment by assuming that the patient is in a standard position relative to the pressure sensor array. In other embodiments, this association is performed by identifying a boundary between pressure sensors that are providing pressure values and pressure sensors that are not providing pressure values and using the boundary to define an outline of where the patient’s body contacts the pressure sensor array. Anatomical locations are then estimated from this outline.

In step 1502, pressure sensors that are positioned at target anatomical locations are identified. Each target anatomical location is a location where a pressure change is expected if the implanted lead is correctly positioned. At step 1504, a distance is determined between the pressure sensors at the target anatomical location and the closest group of pressure sensors identified in step 1314 for any of the contacts. This distance may be measured from the center of the target pressure sensors to the center of the group of pressure sensors or may be measured between the edges of the target pressure sensors and the edges of the group of pressure sensors.

At step 1506, the distance is compared to a threshold distance to determine whether the closest group is near enough to the target location to be acceptable. If the closest group of pressure sensors is not within the threshold distance of the target location, the stimulation lead is repositioned within the patient at step 1508.

Since each contact provides its own groups of pressure sensors, selecting the closest group involves selecting the contact that produced the closest group. If the closest group with a measured response is near enough to the target location to be acceptable, the contact that produced the closest group is recorded at step 1510.

A response curve is then generated using the recorded contact at step 1512.

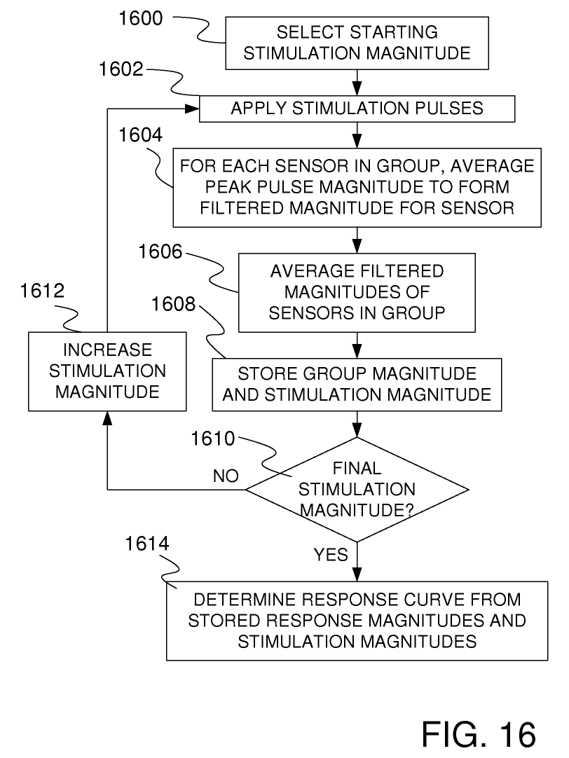

FIG. 16 provides a flow diagram of a method of generating a response curve at step 1512. At step 1600, a starting magnitude for the stimulation signal is selected and at step 1602, stimulation pulses with the selected magnitude are applied by stimulation controller 1226 to the selected contact. The pressure signal provided by each pressure sensor in the group closest to the target location over a period of time is recorded. Peaks in each pressure signal are then identified and the magnitudes of each peak in the pressure signal are averaged to form a filtered magnitude for each pressure sensor in the group at step 1604. At step 1606, the filtered magnitudes of all the pressure sensors in the group are averaged together to form a single group magnitude. At step 1608, the group magnitude is stored along with the stimulation magnitude or other stimulation parameter.

At step 1610, analyzer 1222 determines if a final stimulation magnitude has been reached. If the final stimulation magnitude has not been reached, the stimulation magnitude is increased by a small amount at step 1612 and the process returns to step 1602 using the new stimulation magnitude. Steps 1602, 1604, 1606, 1608 and 1610 are then repeated. When the final stimulation magnitude has been reached at step 1610, a response curve is determined from the stored response magnitudes and stimulation magnitudes at step 1614.

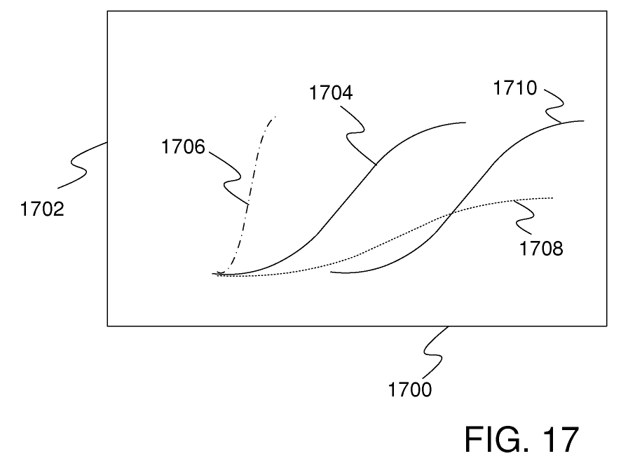

Returning to FIG. 15, after the response curve has been generated at step 1512, analyzer 1222 determines if the produced response curve is acceptable. FIG. 17 provides examples of acceptable and unacceptable response curves with horizontal axis 1700 representing the stimulation magnitude and vertical axis 1702 representing the group magnitude. Graph 1704 of FIG. 17 represents the desired response curve. Response curve 1706 provides an undesirable response curve in which the muscle contractions are overly sensitive to the stimulation signal. Response curve 1708 shows an undesirable response curve in which the muscle contractions are under-responsive to the stimulation signal. Response curve 1710 also provides an undesirable response in which the response curve has the proper shape but requires larger magnitude stimulation signals to be applied than is desirable.

If the response curve does not match the desired response curve at step 1514, the position of the lead is adjusted in the patient at step 1508. If the desired response curve is achieved at step 1514, the magnitude of the stimulation is set at step 1516 based on the response curve. In particular, the desired group magnitude is selected and is applied to the response curve to identify the magnitude of stimulation needed to achieve the desired group magnitude.

In accordance with one embodiment, the position of the lead is adjusted in response to information provided on a display 1250 by processor 1224. This information can include the actual response curve and the desired response curve and the identify of the contact that provided the best response curve. The information can also indicate whether none of the contacts generated a pressure response in the patient.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A system comprising:
a stimulation controller configured to provide a stimulation signal to a patient;
a pressure sensor array, configured to be positioned adjacent the patient and to provide a sensed pressure value; and
a processor configured to associate the sensed pressure value with the stimulation signal.

2. The system of claim 1 wherein the processor is further configured to determine an amplitude or other parameter of the stimulation signal required to obtain a desired change in sensed pressure values.

3. The system of claim 1 wherein the processor associates the sensed pressure value with the stimulation signal by identifying at least one pulse in the sensed pressure values in response to at least one pulse in the stimulation signal.

4. The system of claim 1 wherein the pressure sensor array provides sensed pressure values for a plurality of locations.

5. The system of claim 4 wherein the processor identifies a location that experienced a change in sensed pressure values and determines if the location is associated with proper placement of a stimulation lead in the patient.

6. The system of claim 1 wherein the stimulation controller applies a plurality of different magnitude stimulation signals and the processor is configured to identify a respective sensed pressure value for each magnitude stimulation signal and wherein the processor is further configured to produce a response curve from the plurality of different magnitude stimulation signals and the respective sensed pressure values and to use the response curve to determine if a placement of a stimulation lead in the patient should be changed.

7. The system of claim 1 wherein the stimulation signal comprises a series of pulses and the sensed pressure value comprises a series of sensed pressure values and wherein the processor is configured to use the series of sensed pressure values to remove noise from the sensed pressure values.

8. A method comprising:
placing a pressure sensor array adjacent a patient;
applying a stimulation signal to a lead in contact with the patient;
receiving a signal from the pressure sensor array; and
adjusting at least one of the stimulation signal and a position of the lead based on the received signal from the pressure sensor array.

9. The method of claim 8 wherein the signal from the pressure sensor array comprises location information and magnitude information for pressure values.

10. The method of claim 9 wherein adjusting the position of the lead based on the received signal from the pressure sensor array comprises adjusting the position of the lead based on the location information.

11. The method of claim 8 wherein adjusting a position of the lead based on the received signal from the pressure sensor array comprises applying a plurality of different magnitude stimulation signals to the lead, associating a respective magnitude of the received signal with each of the different magnitude stimulation signals, determining a response curve from the plurality of different magnitudes and the respective magnitudes of the received signal, and using the response curve to determine whether the position of the lead should be changed.

12. The method of claim 8 wherein adjusting at least one of the stimulation signal and a position of the lead based on the received signal from the pressure sensor array comprises:
applying a sequence of stimulation pulses in the stimulation signal;
measuring peak amplitudes of a sequence of pulses in the received signal;
determining an average peak amplitude from the measured peak amplitudes; and
using the average peak amplitude to adjust at least one of the stimulation signal and the position of the lead.

13. The method of claim 8 wherein placing the pressure sensor array adjacent the patient comprises having the patient sit on the pressure sensor array.

14. The method of claim 13 wherein the pressure sensor array is saddle shaped.

15. A system comprising:
an array of pressure sensors, each pressure sensor in the array providing a respective pressure signal;
a stimulation controller configured to provide a stimulation signal to a patient; and
a processor configured to identify a change in one of the respective pressure signals that is due to the stimulation signal.

16. The system of claim 15 wherein the stimulation controller provides the stimulation signal to a lead implanted in the patient.

17. The system of claim 16 wherein the processor is further configured to determine a location of a pressure sensor of the array of pressure sensors that provided the pressure signal that changed due to the stimulation signal.

18. The system of claim 15 further comprising electrical contacts positioned on the patient and an electrical sensor connected to the electrical contacts and capable of detecting the stimulation signal.

19. The system of claim 18 wherein the processor identifies a change in one of the respective pressure signals that is due to the stimulation signal by identifying changes in the respective pressure signal that occurred near times when an electrical pulse was detected.

20. The system of claim 15 wherein the stimulation signal comprises a series of pulses and wherein the processor is further configured to remove noise from the respective pressure signals based on a series of changes in one of the respective pressure signals due to the series of pulses in the stimulation signal.
